(19) United States
(12) Patent Application Publication
(10) Pub. No.: US 2004/0006305 A1
(43) Pub. Date: Jan. 8, 2004

(54) Balloon catheter having an expandable distal end

(76) Inventors: Stephen Hebert, Berkeley, CA (US); Marc-Alan Levine, San Francisco, CA (US)

Correspondence Address: Johney U. Han, Morrison & Foerster LLP, 755 Page Mill Road, Palo Alto, CA, 94304-1018, US

(21) Appl. No.: 10/188,874

(22) Filed: Jul. 3, 2002

(51) Int. Cl. A61M 29/00

(52) U.S. Cl. 604/96.01; 600/3; 604/509

(57) ABSTRACT

A catheter assembly having a balloon integrated at its distal tip. The balloon, while inflated, prevents the back-flow of fluid introduced through the catheter assembly into vessels or ducts in a mammalian subject. The catheter has an inner lumen and a coaxial outer lumen. An inflatable member attached at the distal end of the catheter forms the balloon. The outer lumen provides the channel for inflating the balloon and the inner lumen provides the channel for a guide wire or for use in delivery of fluids or particles. This low profile balloon catheter design allows for high maneuverability and also facilitates the manufacture of small diameter balloon catheters.

### TECHNICAL FIELD OF THE INVENTION

The invention relates to the field of catheters, and more particularly, to balloon catheters with a lumen for delivery of fluids or therapeutics in mammalian vessels or ducts.

### BACKGROUND OF THE INVENTION

Catheters have been used as essential medical instruments for diagnosis and treatment of vascular diseases. In recent years, specially designed catheters used in combination with guide wires have allowed physicians to perform minimally invasive procedures to treat ailments intravascularly. This is done typically by inserting a catheter into the interior of minute vessels, and using a guide wire to advance the catheter along the vessel to reach the point of interest.

Catheters with a balloon at its tip have been used to dilate vessels and clear obstructions within the vessels. Other balloon catheters were designed for temporary or permanent occlusion of vessels. In the treatment of various vascular diseases such as aneurysms, arteriovenous malformations and arteriovenous fistulas, the control of blood flow during treatment is typically necessary. Additionally, a path to provide intervention and treatment is also desirable. To meet this need, balloon catheters with lumens for delivery of fluids or therapeutics have been designed. In other applications, a device that is capable of temporarily seizing blood flow is also desirable. For example, in treatments that utilize local drug delivery techniques, leakage and back-flow of medication during delivery can significantly affect the efficacy of the treatment and also compromise a physician's ability to localize the treatment. In such situations, a device that allows blockage of fluid back-flow in vessels is not only desirable but also necessary for effective application of the medical intervention.

Prior to the present invention, various balloon catheters with lumens for the infusion of fluids have been devised. Examples of such catheters are disclosed in U.S. Pat. No. 6,017,323, issued Jan. 25, 2000 to Chee; U.S. Pat. No. 5,807,328, issued Sep. 15, 1998 to Briscoe; U.S. Pat. No. 5,746,717, issued May 5, 1998 to Aigner; and U.S. Pat. No. 5,700,243, issued Dec. 23, 1997 to Narcisco, Jr., each of which is incorporated herein by reference in its entirety.

Prior catheter designs place the balloon on the outer circumferential surface of the catheter. Such designs resulted in a rising profile around the location of the balloon and also resulted in substantial increases in the diameter of the catheter. This increase in diameter significantly limits where the catheter may be deployed in the vascular system. Vessels distal from the aorta become hard to reach as the diameter of the catheter is increased. In order to secure the balloon on the surface of the catheter, clamps, bonding materials, or other devices are implemented on the surface of the catheter. However, this tends to cause an uneven profile on the surface of the catheter, which makes advancing the catheter in narrow vessels difficult and potentially dangerous. Implementation of a balloon along the shaft of the catheter also tends to significantly limit the flexibility of the catheter around the balloon section and makes the catheter difficult to maneuver.

Therefore, a balloon catheter that is low in profile, with small diameter and highly maneuverable is much desired.

### BRIEF SUMMARY OF THE INVENTION

The present invention relates to a device for the isolated perfusion of therapeutically active substances. The invention may also be used for delivery of diagnostic or therapeutic devices to a desired location within a subject's body. The invention comprises a multi-lumen catheter, which may be used for the complete or partial blocking of the blood flow or fluids around the area of the body to be treated. This interruption or reduction of flow in a blood vessel may permit the administration of locally high concentrations of active substances, which cannot be achieved with conventional techniques without harming the patient. The balloon may also allow physicians to secure the catheter at a desired location within the vasculature for enhancing the ease of deployment of therapeutic and diagnostic devices through an inner lumen of the catheter.

The catheter is designed for insertion into a mammalian hollow body organ. In general, it is to be used in intravascular lumens, but variation of the design are suitable for treatment of any body duct, lumen or space, such as the urinary tract and gastrointestinal tract. The balloon at the tip of the catheter, which is preferably a soft polymer, may also enable atraumatic introduction of the distal tip into a tubular tissue under treatment.

The low profile and highly maneuverable balloon catheter may be used for remote delivery of medication such as chemotherapy agents, anti-angiogenesis proteins or monoclonal antibodies. The administration of such high concentrations of pharmacologically active substances is frequently encountered in medicine. The device may also be used to secure a path in the vasculature for introducing other interventional or diagnostic devices into the body of the subject through the lumen of the catheter.

This may be achieved with a balloon catheter preferably having at least two coaxial lumens, e.g., an inner lumen and an outer lumen, and an inflatable member at the distal end of the catheter covering the outer lumen. When fluid, e.g., saline, water, etc., is injected into the outer lumen, the balloon will inflate and preferably expand outwardly and distally. Alternatively, the balloon may be configured to expand only outwardly, i.e., circumferentially, or only distally. The inner lumen may provide a path for the injection of fluid or it may be use to introduce a second catheter, a guide wire or other device into the body of the subject. One or more radio-opaque markers may be incorporated onto the body of the catheter at predetermined locations, preferably at a distal location. The inflatable membrane may also comprise radio-opaque material so that its expansion and contraction may be monitored visually, e.g., with fluoroscopic equipment.

In an alternative variation, the catheter may be adapted for rapid exchange of guidewires. For example, mechanisms such as ones described in U.S. Pat. Nos. 4,932,413 to Shockey et al. and 6,159,195 to Ha et al., each of which are incorporated herein by reference in its entirety, may be adapted in the present catheter design. Other rapid exchange mechanisms well known to one skilled in the art may also be adapted for enhancing the functionality of the duel lumen catheter.

In another variation, the shaft of the catheter may be designed with variable stiffness. Various methods well known to one skilled in the art may be implemented for fabricating catheters with variable stiffness along the shaft of the catheters.

A guidewire with sensors at its tip, or a miniature catheter with sensors, may be inserted in the lumen of the balloon catheter and used in conjunction with the balloon catheter for delivery and activation of materials and chemicals. Alternatively, sensors may be used for diagnosis or monitoring of treatment.

The disclosed catheter having characteristics of low profile and reduced diameter makes the fabrication of a small diameter balloon catheter with an infusion lumen possible. Furthermore, since the outer edge of the catheter at its distal tip is preferably covered by an inflatable membrane, abrasion caused by the advancement of the balloon in the vasculature is also minimized. Because the balloon is preferably placed at or near the distal tip of the catheter, no supporting device, holes or vents need to be placed along the shaft of the catheter, thus improving the maneuverability of the catheter.

### BRIEF DESCRIPTION OF THE DRAWINGS

The foregoing and other objects, features and advantages of the invention will become apparent from the following description of the invention, as illustrated in the accompanying drawings in which reference characters refer to the same parts through out the different views. The drawings are intended for illustrating some of the principles of the invention and are not intended to limit the invention in any way. The drawings are not necessarily to scale, emphasis instead being placed upon illustrating the principles of the invention in a clear manner.

FIG. 1 is a sectional view of a low profile balloon catheter, showing the balloon in its deflated state.

FIG. 1A illustrates the low profile balloon catheter with the balloon in an inflated state.

FIG. 1B illustrates an alternative variation of a low profile balloon catheter with a balloon that is configured to expand outwardly.

FIG. 1C illustrates an alternative variation of a low profile balloon catheter with a balloon that is configured to expand distally.

FIG. 2 is a semi-transparent view of a low profile balloon catheter with a coil spring placed in the outer-lumen of the catheter to provide structural support to the lumen and the catheter.

FIG. 2A is a cross-sectional view of a catheter body with axially extending wires positioned in the outer lumen of the catheter for structural support.

FIG. 2B is a cross-sectional view of a catheter body with embedded grooves on the outer circumferential surface of the inner-tube.

FIG. 3 illustrates a variation of a low profile balloon catheter with the inner and outer tubes aligned at the distal end of the catheter. The catheter is shown with a semi-annular-shaped balloon attached to its distal end.

FIG. 3A illustrates a variation of a low profile balloon catheter with the inflatable member placed flatly against the opening of the outer lumen.

FIG. 4 is a cross-sectional view of the low profile balloon catheter with valves integrated in the inner-lumen near or at the distal end of the catheter.

FIG. 4A illustrates the frontal view of a low profile balloon catheter with valves integrated in the inner-lumen near or at the distal end of the catheter.

FIG. 5 is a cross-sectional view illustrating an alternative design of a low profile balloon catheter with a distally located infusion section that is constructed of a material different from the material of the elongated body of the catheter.

FIG. 5A is a semi-transparent view of a low profile balloon catheter with a distally located infusion section. The catheter is shown without its inflatable membrane.

### DETAILED DESCRIPTION OF THE INVENTION

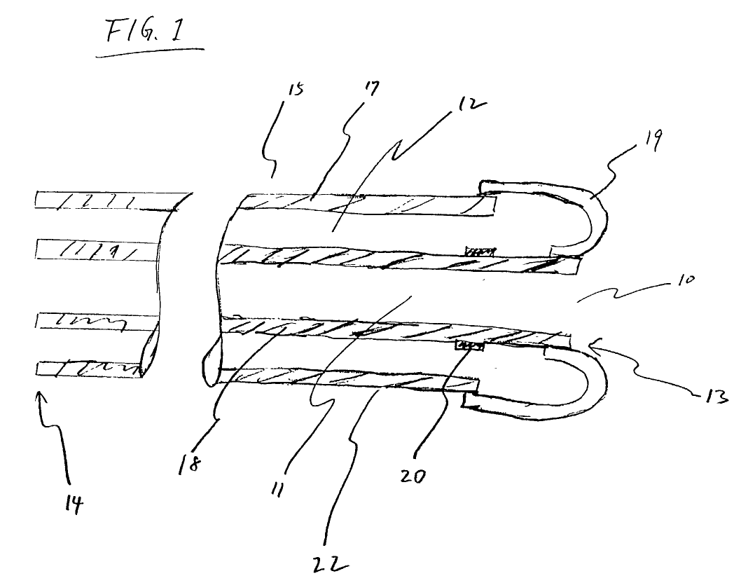

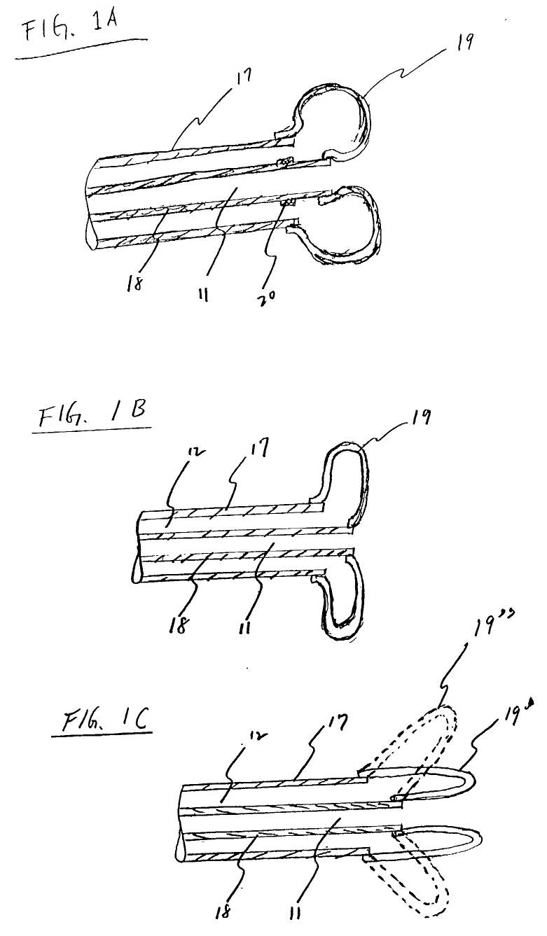

Referring to FIG. 1, the physical structure of a variation on a balloon catheter having a distal port 10 open axially to the inner-lumen 11 of the catheter is disclosed. The catheter generally comprises a distal end 13, a proximal end 14, and an elongated flexible tubular body 17 extending there between. The flexible tubular body is generally comprised of an outer-tube 17, and an inner-tube 18 located inside the outer-tube 17. The inner-lumen 11 of the catheter is defined by the inner-tube 18. The outer-lumen 12 of the catheter is the space defined between the inner 18 and outer-tube 17. A balloon 19 is preferably positioned at or along the distal end 13 of the catheter. The balloon 19 is preferably configured in such a way that it is responsive to the pressure of fluids, e.g., saline, water, or any other biocompatible fluid, which may be delivered in the outer-lumen 12. That is to say that introduction of a fluid into the outer-lumen 12 will inflate the balloon 19 to a predetermined size depending upon the corresponding pressure of the fluid used. For example, the greater the fluid pressure, the greater the inflation of the balloon 19. Furthermore, the fluid, which may be delivered from a proximal location external to the body, may be delivered via a positive pressure pump or manually through a syringe.

Depending on the particular application, the balloon catheter may be of various dimensions. For example, for intercranial catheterization, the catheter body may have an outside diameter within the range of about 0.5 mm to about 1.5 mm. For cardiovascular applications, and catheterization of arteries or veins in other parts of the body, catheter with larger diameter, about 1 mm to about 10 mm, may be used. For most intravascular catheterization the length of the catheter will generally be in the range of about 150 cm to about 200 cm. For example, if the chosen site for treatment is within the brain and the access site is the femoral artery in the groin region, then the length of catheter assembly would be in this range. If the access is through the neck, as would be the case with significantly obese patients, the overall length of the catheter can be much shorter. Catheters with shorter lengths may also be desirable in other applications where penetration may be made close to the target site. For example, catheters designed for urinary tract applications will be much shorter, about 30 cm to about 60 cm may be enough. Other dimensions than those disclosed above and recited elsewhere herein could be readily utilized by those of ordinary skill in the art in view of the disclosure herein to suit particular intended uses of the catheter.

The inner-lumen 12 permits the catheter to track over a guidewire, as is well under stood by those skilled in the art. A guidewire, under the assistance of, e.g., a fluoroscope, may be directed through the tortuous vasculature found in the human body. Once the guidewire is moved over a distance, the catheter may be advanced over the guidewire. Following the placement of the catheter, the guidewire can be removed and the inner lumen may then be used to infuse medication or permit the accomplishment of other diagnostic or therapeutic procedures.

It is preferable that the catheter's inner 18 and outer tubes 17 are both formed of individual single length tubes of a suitable polymer. However, tubing comprised of a composite of sections of various tubing and made of various materials may also be suitable. The inner-tube 18 may comprise a polytetrafluoroethylene tube, or other material that optimizes the slidability of the catheter over a guidewire. The outer-tubing 17 may comprise a polytetrafluoroethylene tube or a polymer and metallic material composite tube, which may provide structural integrity and maneuverability. Other polymer tubing, such as PVC, HDPE or LLDPE tubing may also be used to construct the inner and/or outer tubing. Alternatively, inner 18 and outer tubing 17 may be made from a polymer or polymer composite, and a separate coating or layer may optionally be placed over the tubing to increase tubing lubricity or enhance the biocompatibility. The coating may be deposited on the outer and/or inner circumferential surface of the tubing.

Various other materials may be selected for the construction of the tubing depending on the desired stiffness of the catheter body. Preferred materials are biocompatible plastics such as polyethylene, polypropylene, polyvinylchloride (PVC), ethylvinylacetate (EVA), polyethyleneterephthalate (PET), polyurethanes, polycarbonates, polyamide (such as the Nylons), silicone elastomers, and their mixtures and block or random copolymers. For the more flexible design, softer format (Shore A hardness of 87-95) of the same plastics may be applied. Alternatively, the inner 18 and outer 17 tubes may be comprised of a variety of other materials which are well known in the art of catheter design depending upon the desired physical properties of the finished catheter.

It is also within the scope of this invention to have a variety of regions of differing stiffness along the shaft of the catheter. For example, in treating the vasculature within the soft organs of the body, such as the liver or brain, it is often desirable to have various stages of flexibility within the catheter. That is to say that it may be highly desirable that the proximal section of the catheter body be stiffer than the midsection which in turn may be stiffer than the more distal section adjacent the balloon section at the distal tip. Depending of the particular application, sections of the catheter may be enhanced with relatively stiff materials such as polyamide. Alternatively, the shaft may be made up generally of polymers, layers of polymers, or stiffeners such as coils or braided members to provide torqueability and appropriate stiffness. Tubing with, e.g., integrated ribbons, filaments and or braids, may also be used to construct catheters with the desired stiffness. Structurally enhanced tubing may enhance a variety of desirable properties, such as pushability, torqueability, and resistance to kinking or compression by radially inwardly directed forces.

In an alternative design, an anchoring device may be provided in the distal region of the catheter to secure the position of the outer tubing relative to the inner tubing. In yet another variation, connective members may be placed along the lumen of the catheter for securing the position of the outer tubing relative to the inner tubing.

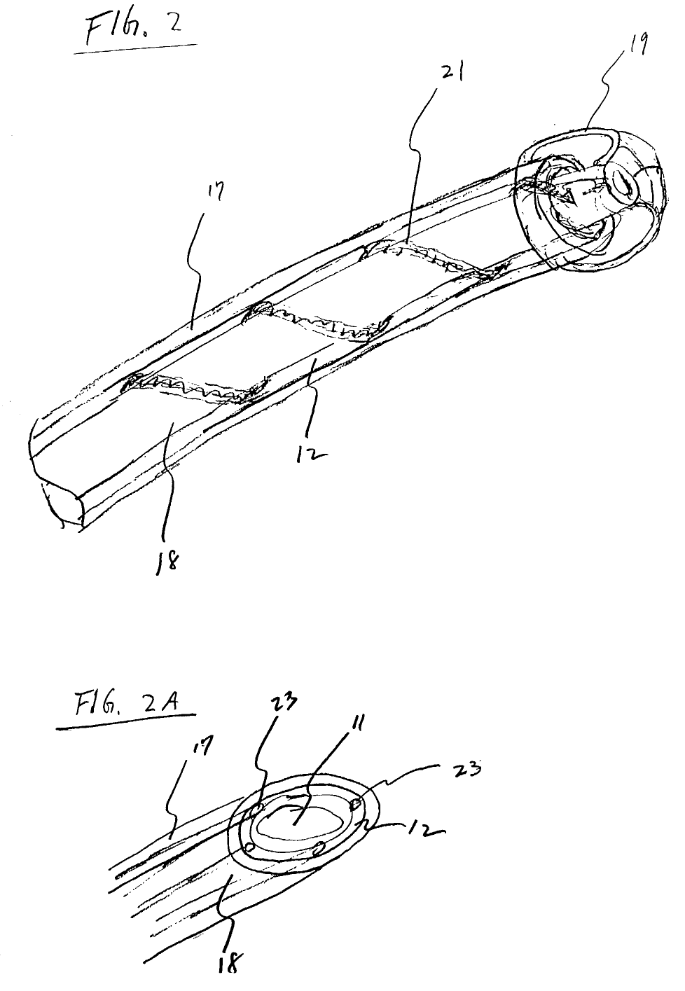

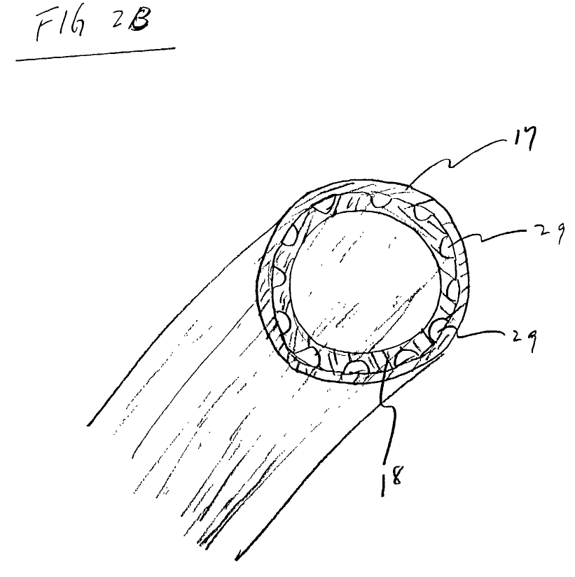

Structural enhancement of the catheter may also be provided within the outer-lumen 12. For example, an axially extending stiffening member, such as a coil spring 21, may be placed between the inner-tube 18 and the outer-tube 17 to provide structural support for the outer lumen. The coil spring 21 placed in the outer lumen 12, as shown in FIG. 2, may also enhance a variety of desirable properties, such as pushability, torqueability, and resistance to kinking or compression by radially inwardly directed forces. Adequate space is preferably provided between the coil 21 so that fluids may advance within the outer lumen 12 from the proximal end to the distal end of the catheter. For example, a continuous coil spring may be placed in the outer-lumen 12 of the catheter in a helical form so as to provide structural support to the catheter while allowing fluids to flow through the outer-lumen 12 from the proximal end 14 to the distal end 13. The coil 21 may be formed integrally along the inner surface of outer tube 17 or along the outer surface of inner tube 18. Alternatively, it may also be formed integrally attached to both inner 18 and outer tubes 17. Furthermore, the coil 21 may be wound about the catheter with a uniform pitch or it may be wound with a variable pitch. For instance, in sections of the catheter where greater stiffness is desirable, such as the proximal portion, the coil 21 may be wound with a higher pitch than compared to the distal portion of the catheter, where the coil 21 may be wound with a lower pitch to result in a more flexible section.

In another variation, metallic ribbons are placed in the outer lumen 12 in place of the coil spring 21 to achieve the desired functionality. The term “ribbon” may include cross-sectional shapes or areas such as a rectangle, square, oval, semi-oval, etc. Suitable nonmetallic ribbons or wires include materials such as those made of polyaramindes (Kevlar), polyethylene terephthalate (Dacron), polyester (e.g. Nylon), or carbon fibers may also be used. Depending upon the intended use of the catheter, alternative stiffening structures may be employed. For example, one or more axially extending stiffening members 23, such as wires or rods, can be provided between the inner 18 and outer tube 17 to enhance desired physical property, as shown in FIG. 2A, preferably so long as a path for fluid flow from the proximal end to the distal end is preserved. The ribbon and other axially extending stiffening materials may have holes or pores defined along its length to minimize flow resistance along the outer-lumen 12 of the catheter. Optimizing the physical property of a particular catheter can be readily done by one of ordinary skill in the art in view of the disclosure-herein for any particular intended use of the catheter.

An alternative design variation may separate the outer lumen into at least two separate channels. Coil springs, ribbons or other flexible materials may be placed in the outer lumen of the catheter to separate the lumen into two separate channels that directs fluid-flow from the proximal end of the catheter to the distal end where the balloon is located. One variation of the design places two helical-shaped coil-spring inside the outer-lumen 12 of the catheter parallel to each other. The two coil-springs can separate the outer lumen into at least two separate channels and at the same time provide structural support to the outer lumen and the catheter body.

In another variation, the outer surface of the inner-tube has grooves and/or ridges integrated on the outer circumferential surface. The grooves and/or ridges on the outer surface of the inner-tube are constructed in such a way that when the outer-tube is placed over the inner-tube, one or more channels/lumens are formed between the outer-tube and the inner tube. These channels/lumens are configured so that fluid may flow from the distal end of the catheter to the proximal end of the catheter through these channels/lumens. FIG. 2B illustrates a cross-sectional view of a catheter body that is constructed with an inner-tube with embedded grooves on the outer circumferential surface of the tube. The grooves 29 form the channels/lumens for fluid flow along the axis of the catheter body. Alternatively, the grooves or ridges may be positioned on the inner surface of the outer tube. It is also feasible to construct the channels/lumens from combinations of grooves and ridges located on both the inner surface of the outer-tube and the outer surface of the inner tube.

In another variation, a tubular jacket is placed over the outer tube to provide a smooth exterior surface 22. The outer jacket may be comprised of a heat shrinkable polyolefin such as polyethylene. The outer tubular jacket preferably extends through the length of the catheter.

FIG. 1 illustrates the distal end 13 of the balloon catheter assembly where the balloon 19 may be attached. The balloon 19 preferably covers the coaxial opening of the outer-lumen 12. The balloon 19 is shown in its deflated state. The inner-tube 18 of the catheter may extend beyond the outer-tube 17 of the catheter, and the balloon 19 may be attached to the distal end 13 of the inner-tube 18 and the distal end of the outer tube 17. The balloon 19 is preferably configured in such a way that it is responsive to the pressure of the fluid which may be pumped in the outer-lumen 12. That is to say that introduction of fluid into the outer-lumen 12 and the buildup of pressure within the outer-lumen 12 will preferably inflate the balloon 19 to a desired size as determined by the amount of fluid pressure. Buffered saline, distilled water and other liquids may be used to inflate the balloon. The balloon may also be inflated by air or non-toxic gases, although this may be less preferable. FIG. 1A shows the balloon 19 in its inflated state. The balloon shown in FIG. 1A expands both outwardly and distally. In other design variations, the balloon may be configured to expand more outwardly, i.e. circumferentialy, as shown in FIG. 1B, or more distally, as shown in FIG. 1C.

In an alternative design, the balloon may expand at an angular direction, between the ninety-degree angular expansion shown in FIG. 1 B and the zero-degree angular expansion 19′ shown in FIG. 1C, the angle being measured relative to the catheter longitudinal axis. As an example, the balloon 19″ is shown to expand at a forty-five degree angle, forming a funnel shape at the distal end of the catheter. The funnel shaped balloon facilitates the collection of materials and fluids in the vessels when suction is applied at the proximal end of the catheter and negative pressure is created in the inner lumen of the catheter. The angled balloon preferably forms a funnel with an opening having an angle between about sixty-degree to about ninety-degree. The balloon may be preformed such that when it expand, it will expand at a predefined angular direction. Various other techniques well known to one skilled in the art may also be used to fabricate balloons that adapt to particular funnel shapes when expanded.

The balloon 19 itself, depending upon the use to which the catheter is to be placed, may be either a compliant balloon or one having a predetermined fixed diameter. The balloon 19 may be made of a variety of materials depending upon the use to which the catheter is applied. For “non-angioplasty” applications, the balloon is desirably produced from elastomeric materials. For angioplasty applications, it may be produced of materials such as polyethylene. Polyethylene balloons are not elastomeric and merely collapse and folds when not inflated. An elastomeric balloon, on the other hand, is simply inflatable. It need not be folded. Such elastomeric balloons are suitable for use in the occlusion of vessels, for placement of medication or diagnostics and for dilatation of vasospastic vessels, e.g., vessels which open with minimum radially applied force. The catheter design described herein is suitable for any size of catheter. This particular catheter design allows the axial length of the balloon to be minimized if so desired. The axial length of the balloon (dimension of the balloon along the axial direction of the catheter) may be as short as the thickness of the inflatable membrane (e.g. when the distal ends of the inner and outer tubing are aligned) and the non-expanded diameter may approximate the diameter of the outer-tube 17. For instance, preferable inflated balloon diameters may range anywhere up to 6 mm, depending upon the type of application, with lengths ranging anywhere up to 40 mm. The elastomeric balloon may be made of a material such as natural or synthetic rubbers, silicones, C-flex, polyurethanes, and their block or random copolymers. Another useful class of materials is elastomeric urethane copolymers, e.g., polyurethane/polycarbonate thermoplastics.

Suitable adhesives may be used to seal the balloon 19 against the outer-tube 17 and the inner-tube 18. Alternatively, the balloon may be attached to the tubular body of the catheter with RF welding. Other methods, such as thermal bonding, solvent boding, adhesives, welding, or any of a variety of other attachment techniques known in the art may also be used to secured the balloon 19 to the catheter. In addition, a hydrophilic coating over the balloon 19 is sometimes desirable.

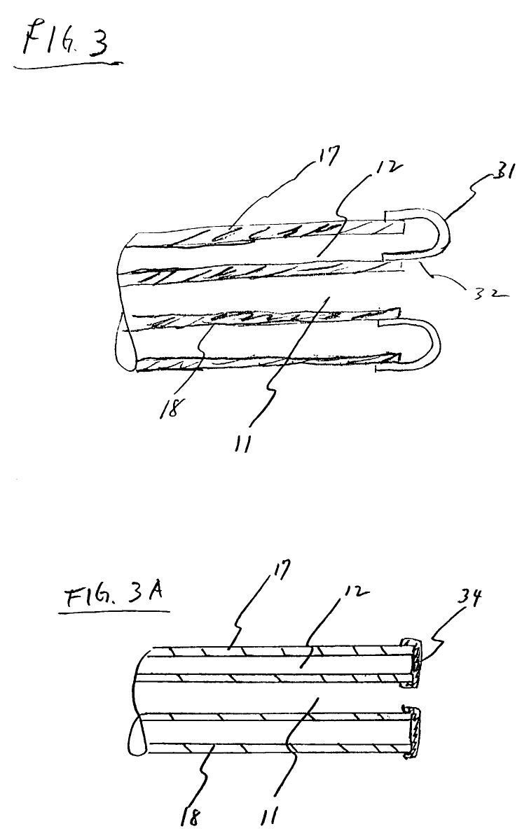

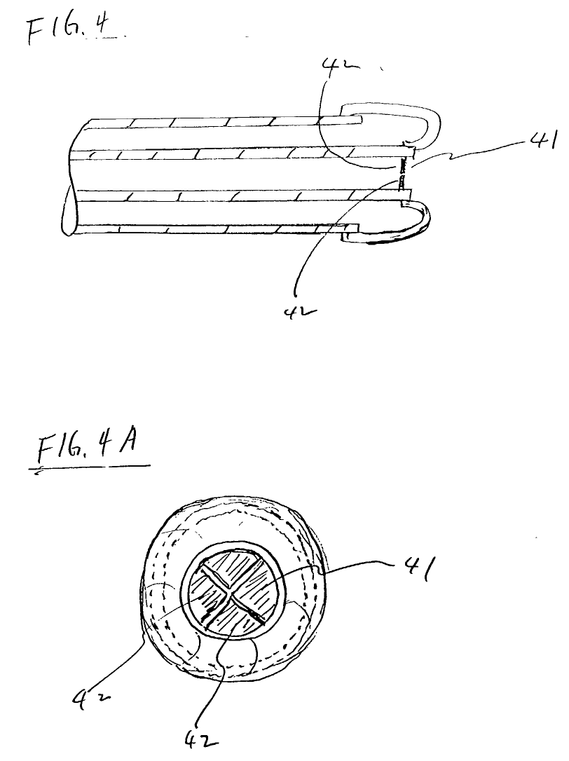

FIG. 3 shows a variation of the above design where the inner 18 and outer tubing 17 are aligned at the distal end 13 of the catheter. A semi-annular or half-donut-shaped membrane 31 is adapted to the distal end of the catheter. The membrane is attached to both the inner tube 18 and the outer tube 17 and a seal is formed. The semi-annular-shaped membrane 31 may be structurally enhanced in the inner core 32 so that when it is fully inflated, it expands radially outwardly and does not constrict the axial opening. An inflatable member 34 may also be placed flatly across the opening of the outer lumen 12 as illustrated in FIG. 3A.

Although both FIG. 1 and FIG. 2 show the membrane attached to the outer wall of the inner tube 18 and the outer wall of the outer tube 17, it is understood that the membrane may also be attached to the inner wall of the tubes instead. Other combination of attachment, including attaching the membrane to the inner wall of the inner tube 18 and the outer wall of the outer tube 17 or attaching the membrane to the outer wall of the inner tube 18 and the inner wall of the outer tube 17, are also contemplated by this invention. It is also feasible to attach the membrane to the inner wall of the inner tube 18 and the inner wall of the outer tube 17. In a variation of the present design, the semi-annular shaped membrane 31 may have predefined sleeves at the edge of the membrane that is capable of securing the balloon 19 to the distal end of the inner 18 and outer tubes 17.

As seen in FIG. 1 and FIG. 2, the deflated balloon 19 in each variation has a very low profile, which approximates the profile of the catheter assembly just adjacent to the balloon 19 itself. Such a design may provide enhanced maneuverability and minimizes tissue abrasion caused by the catheter as the catheter is advanced inside narrow and complex vasculature.

The balloon 19 may have an inner member such as a braid or coil to enhance the structure of the balloon when it is inflated. The balloon 19 may be either elastomeric or of a fixed size. Polymeric or metallic material may also be integrated with the balloon membrane or alternatively adapted as an inner member to define the size of the balloon if so desired.

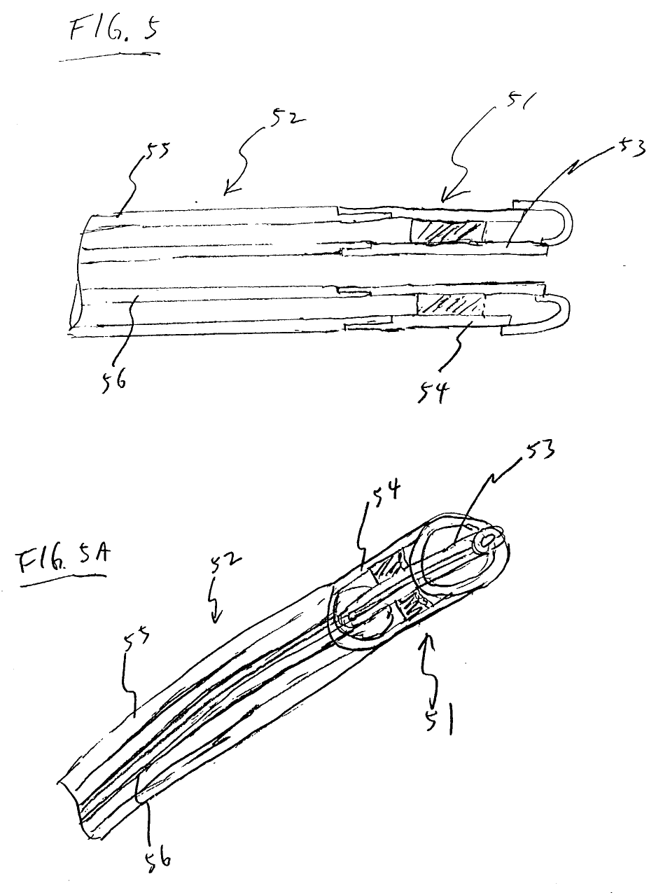

Any of the catheters of the present invention may additionally be provided with a valve 41 at or near the distal end 13 of catheter inside the inner-lumen 11. FIG. 4 illustrates one variation of such a design. In this illustration, the valve comprises four coactive leaflets. Leaflets 42 cooperate in a manner that will be well understood to those skill in the art, to permit the passage of a guidewire (not illustrated) there through and resiliently return to a relatively closed configuration as illustrated, following withdrawal of the guidewire. The leaflets 42 will also open when the pressure inside the inner-lumen exceeds a predefined threshold. However, the leaflets 42 may be designed such that they will not open in response to increases in pressure outside the catheter. Leaflets 42 are preferably constructed from a relatively resilient material to provide a bias to return to the closed configuration. Preferably, the bias provided by leaflets will be sufficient to substantially resist the fluid pressure developed outside the inner-lumen 11 after infusion of the medication into the vascular system.

Leaflets 42 can be constructed in any of a variety of manners, such as by integral construction with the wall of distal segment, or by separate formation and subsequent attachment to the distal segment. For example, leaflets 42 may be separately molded or punched from sheet stock of a compatible material, such as a high density polyethylene, and thereafter adhered to the distal segment such as by thermal bonding, solvent bonding, adhesives, welding, or any of a variety of other attachment techniques known in the art. Alternatively, the polymer chosen for use as a valve can be molded as a tube containing a closed septum. This molded unit can be heat fused or bonded onto the catheter tubing. The septum can then be cut to produce the valve leaflet described previously. The number of leaflets can be varied as desired to accommodate catheter design and manufacturing issues. For example, two or more coactive or cooperative leaflets may also be used.

Depending upon the desired functionality of the catheter, a unidirectional valve or bi-directional valve may be adopted in the inner-lumen 11 to modulate fluid flow. A self-healing membrane or plug may also be placed in the inner lumen to form a valve 41. Alternatively, the valve 41 may be constructed to accommodate a relatively small fluid flow even in the “closed” position to prevent stagnation in the vessel at the distal end of the catheter as will be understood to one skill in the art. Other valve mechanisms well known to one skilled in the art that are of suitable size and material may also be appropriate.

Preferably, the catheter is further provided with a radio-opaque marker 20, such as a band of platinum, palladium, gold or other material known in the art. These circumferential radio-opaque bands act as markers 20 under, e.g., fluoroscopic X-ray visualization. The radiopaque marker 20 may be provided in the form of a metal ring, which may be positioned within the outer tubular jacket prior to application of a heat-shrinking shell to secure the radiopaque maker 20 within the outer tubular jacket. Alternatively, a radio-opaque band 20 may be secured around the inner tube 18, as seen in FIG. 1. Various attachment devices, bonding materials and adhesives well known to one skilled in the art may be used to secure the maker around the inner and/or outer tube. The radio-opaque markers 20, which can be detected by X-ray, may be placed on suitable points on the catheter in order to permit an exact positioning of the catheter within the blood vessel under image converter monitoring.

In another variation, the balloon 19 is bordered by X-ray contrast markings on the edges of the balloon 19. The contrast marking may be obtained by incorporating radio-opaque pigment in the polymer material of the catheter tubing at the desired points. The radio-opaque balloon may allow for the physician to monitor the inflation and deflation of the balloon during a procedure. Alternatively, an inner member such as braid or coil may be adapted inside the balloon to provide radio-opacity.

It is additionally possible to fill the balloon 19 with a radio-opaque fluid for expanding the balloon and in this way to further improve the monitoring of the position. The X-ray contrast medium may be introduced through the outer-lumen 12 of the catheter.

Alternatively, if the catheter is designed for the local delivery of radiation, such as for radiation therapy, it may be preferable to use materials that are radio-transparent in fabricating the balloon.

Another variation of the present invention implements a distally located infusion section 51 with an inflatable balloon 19 that is connected to the elongated body 52 of the catheter. FIG. 5 illustrates one particular design of this variation. The distal infusion head 51 may be comprised of a short tube 53, which is preferably between about 3 mm to about 30 mm, and more preferably between about 5 mm to about 10 mm. The short tube 53 is coaxially placed in another tube 54 that is larger in diameter than the short inner tube 53. The inner tube 53 may be secured to the outer tube 54 through a variety of fastening methods, e.g., adhesives. Additional members, e.g., spacers, etc., may be placed between the inner tube 53 and the outer tube 54 to fix the position of the two tubes. In one particular design, the inner tube's 53 distal end may extend beyond the outer tube a short distance, e.g., several millimeters. However, the inner tube 53 may be of various lengths relative to the outer tube 54, depending on design needs. The inner tube 53 of the infusion section may also be aligned with the outer tube 54 of the infusion section at the distal end of the catheter. It is preferable that the inner tube extend beyond the outer tube by between 0 mm to about 15 mm, and more preferable between 0 mm to about 10 mm. The infusion section 51 may be constructed of polymer or metal. Depending on the particular application, materials that are stiff and non-compliant or materials that are flexible and compliant may be selected for the fabrication of the infusion section.

The elongated body 52 of the catheter may comprise two tubes with one positioned inside the lumen of the other. The distal end of the inner tube 53 may be connected to the proximal end of the infusion section's inner tube 53, and the distal end of the outer tube 51 may be connected to the proximal end of the infusion section's outer tube 51, as seen in FIG. 5A (the balloon which is to be attached at the distal end of the infusion section is not shown for clarity). Spring wiring, metallic ribbon, or polymer ribbon may be placed between the inner 56 and outer tubes 55 of the elongated body 52 to provide structure support. The elongated body of the catheter 52 may be comprised of at least two coaxial tubes as described above, or alternatively an elongated body 52 with at least two channels may be adapted to the infusion section to form a catheter. At lease one channel is connected to the outer lumen of the infusion section for inflating the balloon and a second channel connected to the inner lumen of the infusion section for delivery of medication or chemicals through the catheter.

### EXAMPLE 1

The use of a low profile balloon catheter assembly to facilitate local drug delivery is described in this example. A guidewire is inserted into the femoral artery in the groin region of the subject and advanced into the vascular system towards the desired treatment location. Once the guidewire has been advanced intravascularly over a distance, the catheter may be slid along the guidewire. When the distal end of the balloon catheter has reached the target location, the guidewire may be removed and a fluid, e.g., buffered saline, may be injected into the outer lumen of the tubular catheter to inflate the balloon. The inflated balloon may be inflated to temporarily prevent blood flow in the artery within which the catheter has been advanced. Medication such as chemotherapy agents, monoclonal antibodies or anti-angiogenesis proteins may be injected into the inner lumen of the catheter from its proximal end. The medication will travel through the catheter and exit the catheter at its distal end. The inflated tubular balloon at the distal end of the catheter may prevent back-flow of the medication and keep the medication localized up stream away from the catheter assembly. Once the delivery of the medication is completed, the pressure inside the outer-lumen may be released and the balloon deflated. After the deflation of the balloon, the catheter may then be withdrawn from the subject.

### EXAMPLE 2

The catheter is inserted into the subject as described in Example 1. After the catheter is secured at the desired intravascular location by inflating the balloon, a guidewire with a desired sensor or device attached to it, may be delivered to the site of interest through the lumen of the catheter. The guide wire may have a CCD camera, optical sensors, chemical sensors, a pH sensor, a glucose electrode or semiconductor sensors attached to its distal tip or along its shaft.

### EXAMPLE 3

In this example, the application of a low profile balloon catheter assembly for localized delivery of radiation for radiation therapy is described. As described in Example 1 the catheter is inserted into the subject with the assistance of a guidewire. Once the catheter is desirably positioned, radioactive material such as a chemical isotope may be injected into the outer lumen of the catheter to inflate the balloon. Of course in such an application it would be preferable that the balloon be constructed of radio-transparent material. The balloon keeps the radioactive material contained in the body of the catheter, as the balloon is inflated; radiation dosage also increases since radioactive material accumulates inside the balloon. In an alternative approach, a predetermined amount of the radioactive material may be injected first, followed by a non-radioactive material to push the radioactive material distally to the distal tip of the catheter to inflate the balloon. The radioactive material may be carried by a liquid or gel-like substance. Once the treatment is completed, suction may be applied at the proximal end of the catheter to remove the radioactive material from the lumen of the catheter. With the above-described procedure, the gamma radiation released by the isotope is largely confined to the tissue immediately adjacent to the balloon thus minimizing unnecessary radiation exposure to healthy tissues in other parts of the body.

Optical fiber may optionally be placed in the inner-lumen of the catheter for monitoring local tissue during treatment. Alternatively, a sensor, such as semiconductor radiation detector, may be attached to the tip of a guide wire and inserted up the catheter lumen for monitoring local radiation dosage. Other sensors may also be positioned at the target site as described above to monitor local physiological conditions.

The local delivery of radiation, as described above, may also be utilized for local activation of chemicals. The chemical agents to be activated by radiation may be delivered through the inner lumen of the subject or alternatively injected directly into the subject.

This invention has been described and specific examples of the invention have been portrayed. The use of those specifics is not intended to limit the invention in anyway. Additionally, to the extent there are variations of the invention, which are within the spirit of the disclosure or equivalent to the inventions found in the claims, it is our intent that this patent will cover those variations as well.

We claim the following:

1. A balloon catheter for use in mammalian hollow body organs comprising:
a first elongated tubular body with a distal end, a proximal end and a lumen defined therebetween;
a second elongated tubular body with a distal end, a proximal end, and a lumen defined therebetween, wherein said second tubular body is positioned within the lumen of said first elongated tubular body; and
an inflatable member connected to the distal end of said first tubular body and the distal end of said second tubular body.

2. A balloon catheter as in claim 1 wherein the distal end of said second elongated tubular body extends beyond the distal end of said first elongated tubular body.

3. A balloon catheter as in claim 1 wherein said inflatable member is adapted to expand radially from the catheter.

4. A balloon catheter as in claim 1 wherein said inflatable member is adapted to expand distally along the axial direction of the catheter.

5. A balloon catheter as in claim 1 wherein said inflatable member is adapted to expand both radially and distally from the catheter.

6. A balloon catheter in claim 1 wherein said inflatable member is adapted to expand angularly relative to a longitudinal axis defined by the catheter.

7. A balloon catheter as in claim 1 wherein said inflatable member is radiopaque.

8. A balloon catheter as in claim 1 further comprising a radiopaque marker disposed along the balloon catheter proximally of the inflatable member.

9. A balloon catheter as in claim 8 wherein said radiopaque marker is a band.

10. A balloon catheter as in claim 9 wherein said radiopaque marker band is located near or at the distal end of said first tubular body.

11. A balloon catheter as in claim 9 wherein said radiopaque marker band is located near or at the distal end of said second tubular body.

12. A balloon catheter as in claim 1 wherein said inflatable member is inflatable with saline.

13. A balloon catheter as in claim 1 wherein said inflatable member is inflatable with a radiopaque fluid.

14. A balloon catheter as in claim 1 further comprising a coil spring positioned between said first elongated tubular body and said second elongated tubular body.

15. A balloon catheter as in claim 1 further comprising a ribbon positioned between said first elongated tubular body and said second elongated tubular body.

16. A balloon catheter as in claim 15 wherein said ribbon is helically configured about said second elongated tubular body.

17. A balloon catheter as in claim 1 further comprising a plurality of axially extending stiffening members positioned between said first elongated tubular body and said second elongated tubular body.

18. A balloon catheter as in claim 1 wherein said inflatable member comprises an elastomeric material.

19. A balloon catheter as in claim 1 wherein the distal end of said second elongated tubular body and the distal end of said first elongated tubular body are aligned.

20. A balloon catheter as in claim 1 further comprising:
a valve positioned within the lumen of said second elongated tubular body at the distal end of said second elongated tubular body.

21. A balloon catheter as in claim 20 wherein said valve is a unidirectional valve.

22. A balloon catheter in claim 20 wherein said valve is comprised of a plurality of leaflets.

23. A balloon catheter for use in mammalian hollow body organs comprising:
an elongated flexible tubular body with a distal end and a proximal end, said elongated flexible tubular body defining a first channel extending from the proximal end to the distal end of said elongated flexible tubular body, and said elongated flexible tubular body further defining a second channel extending from the proximal end to the distal end of said elongated flexible tubular body;
an inflatable member adapted to a tip at the distal end of said elongated flexible tubular body with said inflatable member in fluid communication with said first channel.

24. A balloon unit for use in mammalian vessels comprising:
a first tubular body with a distal end, a proximal end and a lumen defined therebetween;
a second tubular body with a distal end, a proximal end and a lumen defined therebetween, wherein said second tubular body is concentrically positioned within the lumen of said first tubular body and wherein an annular space is formed between the second tubular body and the first tubular body, and wherein the distal end of said second tubular body extends beyond the distal end of said first tubular body; and
an inflatable member attached to the distal end of said first tubular body and the distal end of said second tubular body, wherein said inflatable member is adapted to inflate when a pressure in the annular space is increased.

25. A process of delivering a chemical locally within a mammalian body, comprising:
a.) advancing a balloon catheter into said mammalian body, wherein said balloon catheter comprises a first elongated tubular body with a distal end, a proximal end and defining a lumen therebetween, a second elongated tubular body with a distal end, a proximal end and defining a lumen therebetween, wherein said second tubular body is positioned in the lumen of said first elongated tubular body, and an inflatable member connected to the distal end of said first tubular body and the distal end of said second tubular body;
b.) inflating said inflatable member; and
c.) injecting said chemical into the proximal end of the lumen of said second tubular body such that the chemical is delivered from the distal end of the second tubular body.

26. A process of delivering radiation locally within a mammalian body, comprising:
a.) advancing a balloon catheter into said mammalian body, wherein said balloon catheter comprising a first elongated tubular body with a distal end, a proximal end and defining a lumen therebetween, a second elongated tubular body with a distal end, a proximal end and defining a lumen therebetween, wherein said second tubular body is positioned in the lumen of said first elongated tubular body, and an inflatable member connected to the distal end of said first tubular body and the distal end of said second tubular body; and
b.) injecting a radioactive isotope into a space defined by said inflatable member.
